(19) United States
(12) Patent Application Publication
(10) Pub. No.: US 2020/0395095 A1
(43) Pub. Date: Dec. 17, 2020

(54) METHOD AND SYSTEM FOR GENERATING AND COMPARING GENOTYPES

(71) Applicant: INSTITUTE FOR SYSTEMS BIOLOGY, Seattle, WA (US)

(72) Inventors: Gustavo Glusman, Seattle, WA (US); Robert Maxwell Robinson, Seattle, WA (US)

(21) Appl. No.: 16/652,527

(22) PCT Filed: Oct. 25, 2018
(86) PCT No.: PCT/US18/57460

§ 371 Date: Mar. 31, 2020

Related U.S. Application Data
(60) Provisional application No. 62/577,330, filed on Oct. 26, 2017.

(51) Int. Cl. G16B 20/20; G16B 50/20; G06K 9/62; G06N 3/12; G06N 7/00

(52) U.S. Cl. CPC G16B 20/20; G16B 50/20; G01N 2035/00158; G06N 3/123; G06N 7/00; G06K 9/6277

(57) ABSTRACT

An ultra-fast solution to the problem of comparing genotypes across genotyping technologies, while preserving privacy, is presented. A method for transforming a standard genotype representation (i.e., a list of alleles associated with IDs representing single nucleotide variants) into a “fingerprint” of the genotype does not require knowledge of the SNP chip technology, and yields fingerprints that can be readily compared to ascertain relatedness between two genotypes even if the genotypes were created using different SNP chip designs. Because of their reduced size, computation on the genotype fingerprints is fast and requires little memory. This enables scaling up a variety of important genotype analyses, including determinations of degree of relatedness, recognizing duplicative sequenced genotypes in a set, and many others. Because the original genotype representation cannot be reconstructed from its fingerprint, the method also has significant implications for privacy-preserving genotype analytics.

### STATEMENT REGARDING FEDERALLY SPONSORED RESEARCH OR DEVELOPMENT

This invention was made with government support under grant NIH 1U54EB020406, awarded by the National Institutes of Health. The government has certain rights in the invention.

### FIELD OF THE DISCLOSURE

The present disclosure relates to new methods and systems for representing genotype data and, more particularly, to new methods and systems for generation and analysis of reduced data sets representing genotype data, and for facilitating analysis of genotype data for comparison and relationship determination.

### BACKGROUND

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

The information in a genome is usually represented as raw genetic sequence and/or as a series of variants that are present in a genome, relative to a reference genome. A personal genome belonging to a human, for example, is represented as a series of variants from a corresponding human reference genome. Commonly, the reference genome is a public resource such as the genome sequence published as part of the Human Genome Project begun around 1990, declared complete in 2003, and improved steadily over the years since the first genome was sequenced.

A genotype is the portion of a genetic makeup of an organism that determines the organism's characteristics, or a subset of those characteristics. In contrast to a whole genome, which may contain thousands, hundreds of thousands, or millions of genetic bases (depending on the organism), genotypes are collections of variants, typically including variants (single nucleotide variants, or SNPs) that are widely distributed (ideally uniformly) and that have some other desirable characteristics (e.g., frequency). In some embodiments, genotypes include variants associated with specific characteristics or genetic markers. Genotypes are typically determined by DNA microarrays of immobilized, allele-specific oligonucleotide probes that are designed to detect and identify alleles of single nucleotide polymorphisms (SNPs), and are therefore referred to frequently as “SNP chips.” Each nucleotide location is generally referred to by an index number that may or may not be cataloged and/or shared among SNP chips designed and used by different organizations. One example of this index number is an rsID number, used in the dbSNP database (https://www.ncbi.nlm.nih.gov/projects/SNP), and used by some SNP chip manufacturers. rsID stands for “reference SNP cluster ID.”

Different SNP chips may target different sets of locations within a particular genome. For example, different SNP chips may be designed to look at different genotypic traits of an organism, or may consider different locations within the genome as relevant to a specific genotypic trait. Additionally, different organizations may use different indexing schemes for the locations of the genomes that are probed. Further, SNP chip designs may change slightly as particular locations in the genome are added and/or removed from those considered relevant to a genotypic trait. As a result of these variations, the comparison of genotypes is complicated, especially when attempting to make comparisons between genotypes determined by different SNP chip versions or technologies.

For all of these reasons, one can have different representations of the genotype information from the same individual, and/or different annotations of the same genotype information. Given two representations of a genotype, determining whether each is derived from the same individual can be a complicated procedure. A related problem is determining whether two genotype representations are derived from related individuals (e.g., siblings, parent and child, etc.). If the genotyping technology (e.g., SNP chip design) differs between genotypes, comparing the genotypes can be a slow, complicated, and error-prone bioinformatic procedure.

Privacy considerations provide a further complication of genotype analysis. While genotype information may be valuable to uniquely identify an individual's genotype, aspects of the genotype information can be associated with the existence of susceptibility to disease, and with a variety of other traits. Applications exist where it would be helpful to retain the ability to identify an individual from a genotype but anonymize or conceal phenotypic associations.

### SUMMARY

A computer-implemented method of generating a genotype fingerprint representing a genotype includes receiving genotype data for an individual. The genotype data includes a plurality of single-nucleotide polymorphisms (SNPs), each having an associated identification number (ID) indicating a corresponding location in a genome of the individual, and each having a defined number of alleles each selected from a defined set of nucleotide types. The method also includes selecting a subset of SNPs from the plurality of SNPs and, for each SNP in the subset of SNPs, determining a value (k) associated with the SNP, the value indicating a column of a data structure, and determined by computing the modulus of the identification number divided by a vector length (L), such that k=ID mod L. For each nucleotide type of the defined set of nucleotide types, the method includes determining a number of occurrences of the nucleotide type in the SNP, the nucleotide type corresponding to a row of the data structure, determining an expected count in the SNP for the nucleotide type, calculating a difference between the number of occurrences of the nucleotide type in the SNP and the expected count in the SNP for the nucleotide type; and adding the difference to a tally at a position in the data structure corresponding to both the column and the row.

In various implementations, the identification number may be an rsID number, the determined number of alleles may be two, the defined set of nucleotide types may consist of A, C, T, and G, and/or the plurality of SNPs may be limited to autosomal SNPs. The vector length may be selected to be less than the number of SNPs in the selected subset of SNPs and/or may be selected such that the number of SNPs in the selected subset of SNPs is at least 20 L.

In some implementations, the method may include computing a cohort average by, for example, analyzing a number of genotypes to determine an average number of occurrences for each nucleotide type for each column for the number of genotypes. The cohort average may be weighted, for each genotype, by the number of alleles contributed by that genotype.

The analysis for a first subset of the plurality of individuals may be determined according to a different SNP chip type or version than the analysis for a second subset of the plurality of individuals, in implementations. The method may result in genotype fingerprints for which relatedness of the two individuals can be determined even when the genotype data for a first of the two individuals was determined by a first SNP chip type or version and the genotype data for a second of the two individuals was determined by a second SNP chip type or version.

In embodiments, a matrix representing the genotype fingerprint may be normalized relative to a reference matrix derived from a set of genotypes. This process may include representing each genotype fingerprint of the set of genotype fingerprints as a corresponding matrix. The process may also include computing, for each position of the matrix, an average and a standard deviation for each matrix in the set of matrices from which the reference matrix is derived, and transforming the matrix by computing a Z-score for each value in the matrix, wherein the Z-score is the value, minus the average, divided by the standard deviation.

The fingerprints may be used to compare genotype information, in implementations. Comparison of genotype fingerprints may include computing a first genotype fingerprint and a second genotype fingerprint, and determining a correlation between the first and second genotype fingerprints. The determination of the correlation may include determining a Spearman correlation coefficient, a Pearson correlation coefficient, or another type of correlation coefficient, and comparing the coefficient to one or more thresholds to determine a relationship between respective samples from which the genotype data of the first and second genotype fingerprints were obtained.

### BRIEF DESCRIPTION OF THE DRAWINGS

FIG. 1 depicts a block diagram of an example computer and system programmed to implement a method or methods in accordance with the present description;

FIG. 2 is a flow chart depicting an example method for generating SNP ID modulo fingerprints in accordance with the present description;

FIG. 3 depicts example data structures that may be generated by the method of FIG. 2;

FIGS. 4A and 4B are flow charts depicting an embodiment of a method for computing an expected value for use in the method of FIG. 2;

FIG. 7 is a flow chart depicting a method for comparing SNP ID modulo fingerprints; and

FIGS. 8 and 9 are graphs depicting the results of a study evaluating the relative strength of the described methods for comparison of genotypes determined using different SNP chip designs.

### DETAILED DESCRIPTION

A novel method and system for generating reduced genotype data sets, and analyzing the reduced genotype data sets to determine various relationships and parameters thereof, is described herein. Stylized as a “fingerprint” of the genotype, the reduced data set is sufficiently distinct for a given individual that it can be compared to another such fingerprint to determine, based on the strength of correlations between the two, if the two are from the same person. Unlike literal finger prints (i.e., the patterns of whorls and ridges in the tips of human fingers), the fingerprints described herein can also be used to determine the degree of relatedness of individuals, as well as other various parameters and characteristics, as will be elaborated upon below. Additionally, unlike other fingerprints or genome and/or genotype data, the presently described embodiments facilitate comparison of genotype data collected by differing designs of genotype assays that analyze single nucleotide polymorphism (SNP), even as assay chip designs evolve to have, for example, higher marker densities. A variety of other, additional advantages of the genotype fingerprints described below will become apparent throughout the remainder of the specification.

As used from this point forward, the term “fingerprint” refers to a set of data representing, for the results of a SNP assay of an individual, a reduced set of data representing a characterization of the numerical identifier data of each SNP (or a selected subset of available SNPs) and the alleles detected at that SNP. Exemplary subsets of SNPs from a genotype assay, from which a genotype fingerprint can be generated, include SNPs from one or a subset of all of the chromosomes from a genome (e.g., the set of autosomes); SNPs from substantial portions of a single or multiple chromosomes, etc. In contrast with some previous methods, in which performing comparisons and analyzing correlations and relationships as described between sequence-based fingerprints (rather than assay-based fingerprints), required comparison between fingerprints made with the same genetic information, e.g., comparison of a whole genome fingerprint with another whole genome fingerprint; or comparison of a whole Chromosome 1 fingerprint with another whole Chromosome 1 fingerprint; and so on, the present embodiments may or may not require that the same subset of SNPs be analyzed in comparison of genotype fingerprints.

The invention described herein is especially useful in the context of analysis of human genotype data. However, it can in principle also be used to generate and analyze/compare genotype data of other animals or even organisms from other kingdoms, e.g., plants or fungi.

The phrase “SNP ID modulo fingerprint” (or the abbreviation SIMF) may refer to a specific type of genotype fingerprint in which the reduced data set represents the distribution of alleles in SNPs stratified at least by the modulus (i.e., the remainder after division) of SNP identifiers (SNP IDs). In contrast with previous methods of analyzing data from genotype assays, the present description does not focus on distances between single nucleotide variants (SNVs) in a genome, or on differences between the identification numbers associated with consecutive SNVs that may be identifiable from SNP data. Instead, this description focuses on embodiments in which allele frequency for SNP data is analyzed with respect to expected allele frequency for the SNP data, without regard to differences in an individual with respect to a reference genome.

As alluded to above, in embodiments the fingerprints are generated, in part, according to the nature of the various SNPs resulting from analysis of a particular genome or portion of genome or exome, for example. As will be understood, the genetic information comprises sequences of four bases: adenine, cytosine, guanine, and thymine (in DNA) or uracil (in RNA) in various orders. In DNA the bases are present as deoxyribonucleosides (deoxyadenosine; deoxyguanosine; deoxythymidine; deoxycytidine). In RNA the bases are present as ribonucleosides (adenosine, guanosine, uridine, cytidine). For purposes of describing the fingerprints herein, the conventional abbreviations for the four bases (A, C, T, and G) are used, with the understanding that T in DNA is operationally equivalent to U in RNA for purposes of generating fingerprints. Many of these bases never, or rarely, vary between individuals in the same population (e.g., ethnicity) or in the same species. For this reason, genotype assays generally analyze portions of a genome that vary with frequency above a particular threshold and that vary in a way that provides useful information about an individual's genotype, specifically the genotype characteristics (e.g., ancestral makeup, markers that are predictive of or correlated with a specific condition, etc.) of interest for the particular assay. Variations among specific positions or groups of positions in the genome are what differentiate one individual from another, and give each individual its unique characteristics and features.

In a genome having two copies of each chromosome, each position in the genome includes two copies (alleles) of the corresponding nucleotide. In mammals (and many other animals), for example, autosomal DNA includes one allele from each of the two biological contributors (e.g., mother and father) to the individual. Thus, at a given position in the genome, an individual will have two associated nucleotides, rendering, where four nucleotide types (e.g., A, T, C, G) are possible, 10 possible combinations of alleles (assuming that the order of the alleles is ignored): AA, AC, AT, AG, CC, CT, CG, TT, TG, and GG. In the present description, the order of the alleles is unimportant, as the methods described herein rely on allele frequency—allele pair AC has the same frequency of allele A and the same frequency of allele C as the allele pair CA. As will be understood, the assays performed for genotype analysis are not always able to detect both alleles at a specific location in the genome, or may detect that a specific allele is deleted or inserted in a particular genome. As a result, some genotype assays may include additional allele identifiers such as “I” for insertions, “D” for deletions, “-” not observed, etc. In at least some of the embodiments described herein, allele identifiers that have no correspondence with a specific nucleotide type are ignored. For instance, in embodiments, SNPs coded as I, D, or - would be ignored. In some embodiments, a SNP that includes a “not observed value” (e.g., G-, -T, etc.) would be reported as homozygous (e.g., GG, TT, etc.).

Generally assays of genotype data are performed using genetic testing arrays on a chip, which arrays characterize the identity of hundreds, thousands, tens of thousands, hundreds of thousands, or millions of SNPs using hybridization analysis of a subject's nucleic acid. Specifically, DNA microarrays (“SNP chips”) of immobilized, allele-specific oligonucleotide probes designed to detect and identify alleles of single nucleotide polymorphisms (SNP). Extensive public libraries of SNP information exist from which it is possible to discern both the identity of SNP alleles (wild type or reference version of a SNP and variants thereof) and the location in the human genome. See, e.g., http[colon-slash-slash]www.ncbi.nlm.nih.gov/snp or http[colon-slash-slash]www.ncbi.nlm.nih.gov/projects/SNP/ or http[colon-slash-slash]www.uniprot.org/database/DB-0013 or http[colon-slash-slash] www.hgvs.org/central-mutation-snp-databases. Thus, SNP arrays can be used to obtain genotype information for the genome to be analyzed.

For each SNP analyzed on a particular SNP chip, the SNP chip generally detects a number of alleles (e.g., two alleles in a biallelic genome such as the human genome). Each SNP is associated with a numerical identifier that indicates the identity of the location in the genome at which the alleles are detected. Thus, to take an overly simplistic example, a particular SNP chip may analyze 10 positions in the genome, and each pair of detected alleles may be associated with a position 1 through 10, and each position 1 through 10 may be associated with a particular location in the reference (e.g., human) genome. The genotype data received from the SNP chip analysis, then, includes, for each SNP analyzed by the SNP chip, an identifier and an output set (e.g., pair) of alleles. For SNP analyses performed by 23andMe, for instance, the identifiers are stylized as rsID numbers and iN* numbers. That is, 23andMe uses both dbSNP identifiers (rsID) and its own set of identifiers (e.g., i[Number]) for variants without rsIDs. Throughout this description, we will use the terms rsID and SNP ID interchangeably to refer to the identifiers associated with particular analysis locations in a genotype analysis. It should be understood that some contemplated embodiments include both the rsID numbers and/or other identifiers (e.g., i identifiers), while other embodiments include only identifiers in the dbSNP database (i.e., rsIDs).

As contemplated herein, in the SNP ID modulo fingerprints, the various analyzed positions in the genome are stratified according to the modulus of the rsID of each (the “SNP ID modulo”). For a given vector length (i.e., a parameter selected according to the various goals and/or intended uses of the SNP ID modulo fingerprints), the modulo function would yield as many “bins” into which allele frequencies could be “sorted.” By way of example and without limitation, for a vector length 20, each rsID could fall into one of 20 “bins” (represented as rows or columns, if the fingerprint is represented as a two-dimensional matrix). Each of the bins corresponds to the remainder of the distance divided by the vector length. For example, for an rsID 10017, the allele frequency data would be in the 17 bin (10017/20 yields a quotient of 500 with remainder 17), while allele frequency data for an rsID 100000 would be in the 0 bin (100000/20 yields a quotient of 5000 with no remainder). Of course, those of ordinary skill in the art will appreciate that the rsID number may be any number selected to consistently identify data relating to the same portion of the genomes being analyzed, including numbers in the hundreds, thousands, tens or hundreds of thousands, etc.

It is possible to apply the methods described herein to configurations that do not employ SNP IDs that have an obvious numerical component. In such configurations, one could convert symbolic or alpha characters to numerical values by, for example, computing the sum of the ASCII codes of each character in the SNP ID. After doing so, the rest of the method would proceed as described herein.

In embodiments, the vector length (denoted as “L”) is selected to maintain the privacy of the individuals' genetic information, such that the resulting genotype fingerprints provide sufficient information about relatedness between individuals, but insufficient information to determine the genotype of each individual. In general, the vector length will be a smaller number than the number of SNP identifiers being analyzed, but will be a large enough number to provide sufficient information about the relatedness of individuals. In various embodiments, the vector length is greater than 500, greater than 1000, greater than 2500, greater than 5000, greater than 10000, between 500 and 10000, between 1000 and 5000, between 2500 and 7500, or between 4000 and 6000. In various embodiments, the vector length is selected such that the number of SNPs being analyzed in an individual genotype is greater than 2 L, greater than 5 L, greater than 10 L, greater than 20 L, greater than 40 L, between 2 L and 50 L, between 5 L and 40 L, between 10 L and 30 L, or between 15 L and 25 L.

Each SIMF then represents, for each nucleotide type, the number of times that the nucleotide type occurs in the genotype analysis as associated with a set of rsIDs having each of the remainders for the selected vector length. Accordingly, in some embodiments, the SIMF is stored and/or represented as a matrix of rrows, where rcorresponds to the number of nucleotide types (and each row corresponds to one of the nucleotide types) and c columns, where c corresponds to the vector length (and each column represents a specific remainder from 0 to one less than the vector length). Alternatively, the SIMF is stored and/or represented as a matrix of rrows, where rcorresponds to the vector length (and each row represents a specific remainder between 0 and the vector length) and c columns, where c corresponds to the number of nucleotide types (and each column corresponds to one of the nucleotide types).

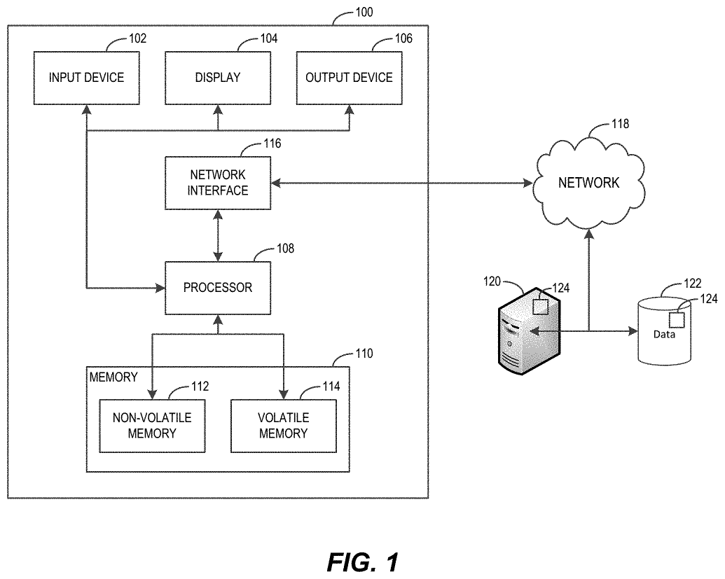

FIG. 1 depicts a block diagram of an example computer 100 and system programmed to implement a method or methods in accordance with the present description. The computer 100 includes one or more input device(s) 102, one or more display device(s) 104, one or more output device(s) 106, and one or more processor(s) 108. Each of the input devices 102 may be any known input device including, without limitation, a pointing device (e.g., a keyboard, a mouse, a track pad, a touch screen, etc.) that allows a user to operate and provide input to the computer 100. The input devices 102 may be internal (as in the case of a laptop computer) or external (as in the case of a USB mouse) to the computer 100, may be hard-wired to or removable from the computer, and may utilize any protocol that facilitates communication between the input device 102 and the processor(s) 108.

Similarly, the display(s) 104 and the output device(s) 106 may be internal (as in the case of a laptop display) or external (as in the case of a USB monitor or a printer), may be hard-wired to or removable from the computer, and may utilize any protocol that facilitates communication between the display(s) 104 and output device(s) 106 and the processor(s) 108. Of course, the displays 104 can utilize any known technology. Additionally, in embodiments, the display 104 may be coupled to and/or integrated with the input device 102, as would be the case in a touch-screen.

As will be understood, the processor(s) 108 may be one or more individual distinct processor packages, may be an integrated multi-core processor in a single package, or may even be multiple multi-core processor packages. The processor(s) 108 are programmed and/or programmable to perform the methods described below, according to machine readable instructions. The machine readable instructions may be stored on one or more memory device(s) 110 comprising any type of tangible, non-transitory media (e.g., magnetic media, solid state media, optical media, etc.) capable of storing data and/or machine-readable instructions executable by the processor 108. The memory 110 may have one or more elements of non-volatile memory 112 (e.g. solid state memory, hard drive, etc.) and one or more elements of volatile memory (e.g., Random Access Memory, or RAM) 114.

The processor 108 may also be communicatively coupled to a network interface 116. The network interface 116 is operable to communicate with one or more network devices via a communication protocol over a network 118. The network interface 116 may be communicatively coupled with the network 118 via any known (or later developed) wired or wireless technology, including without limitation, Ethernet networks, networks adhering to the IEEE 802.11 family of protocols, etc. The network 118, of course, may be any local or wide area network including, for example, the Internet, and may provide access to data (including machine-readable instructions, in embodiments) stored on one or more servers 120 and/or databases 122. In this manner, the processor 108 may retrieve, via the network interface 116 and the network 118, collections 124 of data stored on the servers 120 and/or the databases 122, which collections 124 of data may be updated periodically or in real time, in various embodiments. As a result, and as will be understood in view of the description to follow, the processor 108 may execute the methods described herein using the most recent collections 124 of data available as inputs, and/or may receive new data upon which to operate. Of course, data retrieved via the network 118 may be stored in either or both of the non-volatile memory 112 and the volatile memory 114 for later access and/or manipulation by the processor 108 and/or for comparison to current data stored on the servers 120 and/or the databases 122, in making a determination as to whether the one or more of the collections 124 of data have been updated since they were last retrieved via the network 118. The methods described herein may be stored in the volatile memory 114 and/or in the non-volatile memory 112.

The collections 124 of data stored on the servers 120 and/or the databases 122 may include, by way of example, various genetic sequence data or various genotype data including, by way of example, collections of genotype data (e.g., from SNP array analyses) created using one or more types and/or configurations of SNP chips. The data may include whole genome sequence data, exome sequence data, sequence data for a single chromosome and, in particular, collections of single nucleotide polymorphisms, such as those generated by one or more SNP arrays. In embodiments, the collections 124 of data include collections of genetic sequence and/or SNP data that are generated using the same and/or different technologies as data in other collections or as other data in the same collection, the same and/or different encoding schemes as data in other collections or as other data in the same collection, the same and/or different labeling schemes as data in other collections or as other data in the same collection, the same and/or different reference freezes as other data collections or as other data in the same collection, etc.

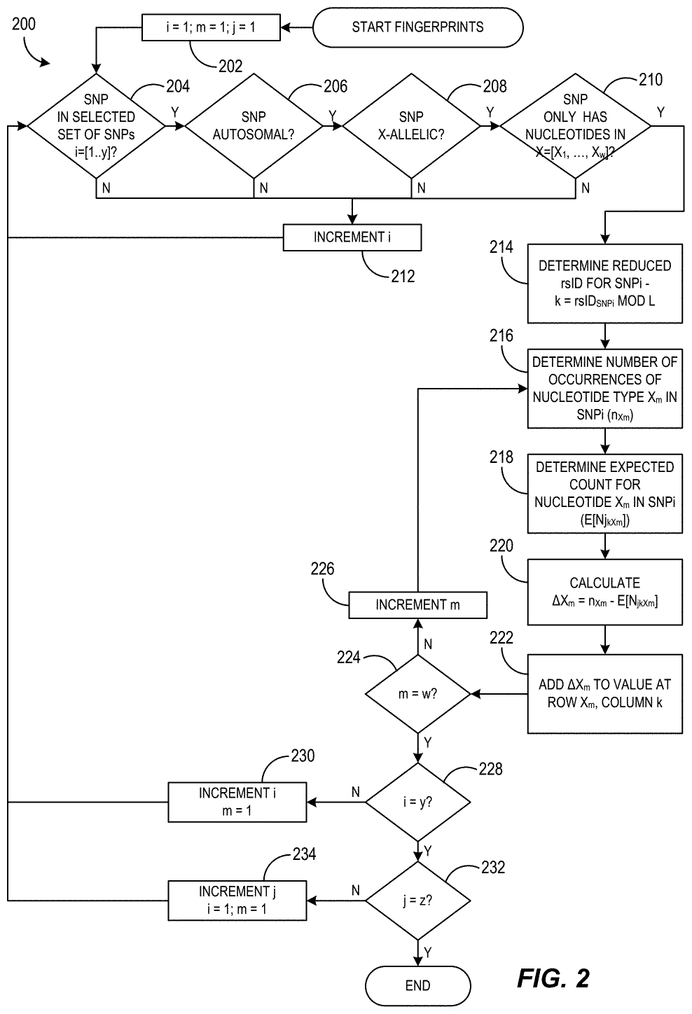

FIG. 2 depicts an embodiment of a method 200 of generating SNP ID Modulo Fingerprints in accordance with the present disclosure. As described, the method 200 is performed by a computer processor (e.g., the processor 108) executing machine-readable instructions stored on a tangible, non-transitory computer readable medium (e.g., the memory 110). In the method 200, all of the available SNPs for a particular individual (e.g., from a single SNP array analysis or, in embodiments, from multiple SNP array analyses using different SNP arrays to determine different sets of SNPs) are located and stored before converting the data of the SNPs to a SIMF. However, in some embodiments of the method 200, the method may exclude non-autosomal SNP data (i.e., the method 200 may be applied only to SNP data for autosomal data), may exclude SNP data having more or fewer alleles than desired (i.e., the method 200 may be applied only to biallelic, triallelic, quad-allelic, etc. SNPs), may restrict the SNPs considered to a specific subset of SNPs available in the SNP data for the individual, and/or may restrict the SNPs considered to those having only a specific set of nucleotide types represented (i.e., the method 200 may exclude SNPs that have allele values other than A, C, T, or G).

In the method 200 depicted in FIG. 2, the processor 108 initializes a variety of variables that are used to recursively perform various analyses of the SNP data. In the example method 200 depicted, the processor 108: initializes a value, i, representing the particular SNP in a set of SNP data with y total SNPs; initializes a value, m, representing the particular nucleotide type of a total w nucleotide types (e.g., m=1, 2, 3, and 4 corresponding, respectively, to nucleotide types A, C, T, and G, with w equal to 4); and initializes a value, j, representing the particular individual for which the SIMF is being generated (block 202), with z individuals total. In the method 200, the values i, m, and j are each initialized to 1, but of course could be initialized to 0 or any number, with appropriate modifications to the remainder of the method, as would be understood by any skilled computer programmer.

In the method 200, the available SNP data are then filtered (blocks 204-210) to limit the analysis to particular SNPs or types of SNPs. For instance, each SNP, SNPᵢ, may be analyzed to determine if the SNP is in a selected set of SNPs for analysis (block 204). That is, a particular implementation of the method 200 may analyze only some of the available SNPs based on the goal of the analysis. If the SNPᵢ is not in the set of SNPs selected for analysis, the processor 108 may increment i (block 212) and begin analysis of the next SNP. If the SNPᵢ is in the set of SNPs selected for analysis, the processor 108 may determine if the SNP is autosomal (block 206). If not, the processor 108 may increment i (block 212) and begin analysis of the next SNP. If so, the processor 108 may determine whether the SNP includes the desired number (e.g., 2) of alleles (block 208). If the SNP, includes more or fewer alleles than desired (e.g., includes 3 or 1 alleles), the processor 108 may increment i (block 212) and begin analysis of the next SNP. Otherwise, the processor 108 may analyze whether the SNPᵢ includes nucleotide types other than those selected (e.g., other than A, C, T, G) (block 210). If the SNP includes additional nucleotide types, the processor 108 may increment i (block 212) and analyze the next SNP. If the SNP includes only the selected nucleotide types, the processor 108 may proceed with the remainder of the method 200.

Of course, the method 200 need not perform all of the filtering steps (blocks 204-210). In various embodiments, some or all of the filtering steps (blocks 204-210) depicted in FIG. 2 may be omitted. Additionally, while FIG. 2 depicts the filtering steps (blocks 204-210) performed on a SNP by SNP basis during the performance of the method 200, some or all of any implemented filtering steps may performed separately on the total set of SNP data prior to implementation of the method for creating the SIMF.

Additionally, while described herein as limited to biallelic, autosomal SNPs having only nucleotide types A, C, T, and G, it should be understood that the method 200, in other embodiments, may include SNP data that is not biallelic (i.e., SNP data where more or fewer alleles are present in each SNP), such as octaploid strawberry, hexaploid wheat, or tetraploid potato, for example. The method 200 may also or alternatively include SNP data that includes fewer or additional nucleotide types (e.g., includes deletion types, insertion types, unknown types, etc.).

In any event, for each SNPᵢ that meets the required criteria established by the filters (blocks 204-210) the SNP is processed to sort its associated data into the data structure representing the SIMF. First, the processor 108 determines into which column the data will be sorted by calculating a reduced rsID, k, for the SNPᵢ:

k=rsID_{SNPi} mod L (Eq. 1)

where k is the column into which the data will be sorted, L is the vector length, and rsID_{SNPi} is the rsID number associated with the SNPᵢ (block 214). The processor 108 then determines the number of occurrences, n_{Xm}, of nucleotide type Xₘ in SNPi (block 216). For instance, where m=1, the processor 108 may determine the number of A nucleotides present in the SNP; where m=2, the processor 108 may determine the number of C nucleotides present in the SNP; where m=3, the processor 108 may determine the number of G nucleotides present in the SNP; etc. By way of example, for a biallelic SNP having alleles AA, the processor 108 would determine that, for m=1, the number of occurrences of nucleotide type Xₘ (e.g., nucleotide type A) is 2 (i.e., n_{Xm}=2, for m=1), while for a biallelic SNP having alleles AG, the processor 108 would determine that, for m=1, the number of occurrences of nucleotide type Xₘ (e.g., nucleotide type A) is 1 (i.e., n_{Xm}=1, for m=1), and for a biallelic SNP having alleles CG, the processor 108 would determine that, for m=1, the number of occurrences of nucleotide type Xₘ (e.g., nucleotide type A) is 0 (i.e., n_{Xm}=1, for m=1). It should be understood that the particular value of m associated with each of the nucleotide types is arbitrary, so long as it is maintained consistently across all fingerprints.

For each nucleotide type, Xm, in the SNPi, the processor 108 will determine (e.g., calculate or retrieve from memory) an expected value, E[n_{Xm}], for the count nxm (block 218). In embodiments, the expected value E[n_{Xm}] is specific to the individual, the nucleotide type, and the column (E[N_{jkXm}]), and is based on cohort averaging. In other embodiments, the expected value E[N_{Xm}] is based on cohort averaging, but is specific only to the column and the nucleotide type (E[N_{kXm}]).

The processor 108 next determines the difference between the number of occurrences of nucleotide type Xₘ (n_{Xm}) and the expected value E[n_{Xm}] for the count n_{Xm}:

ΔXₘ=n_{Xm}−E[N_{Xm}] (Eq. 2)

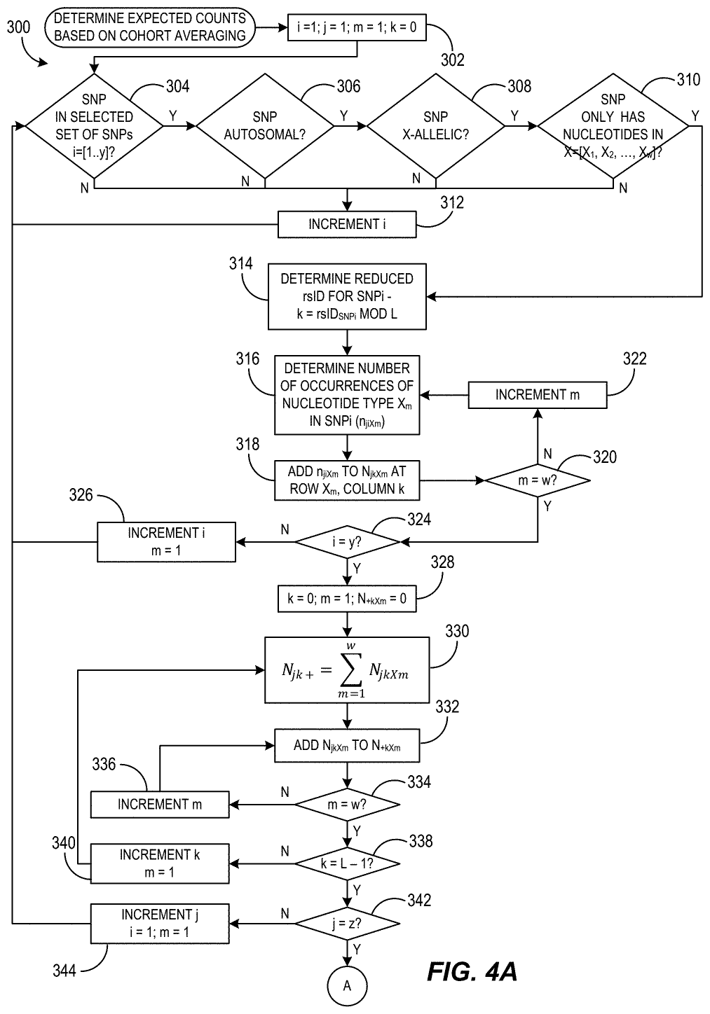

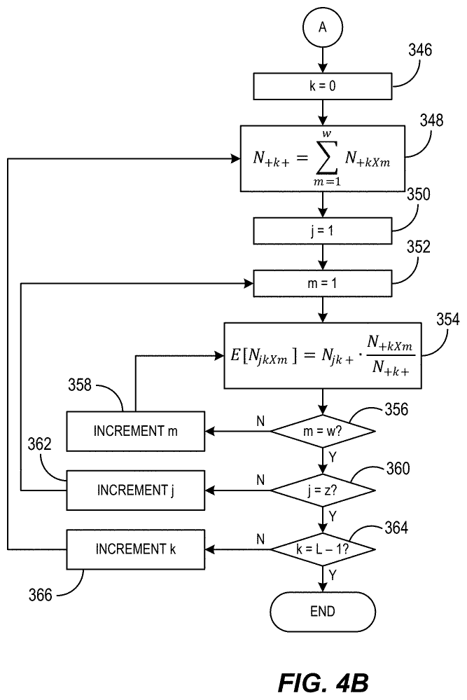

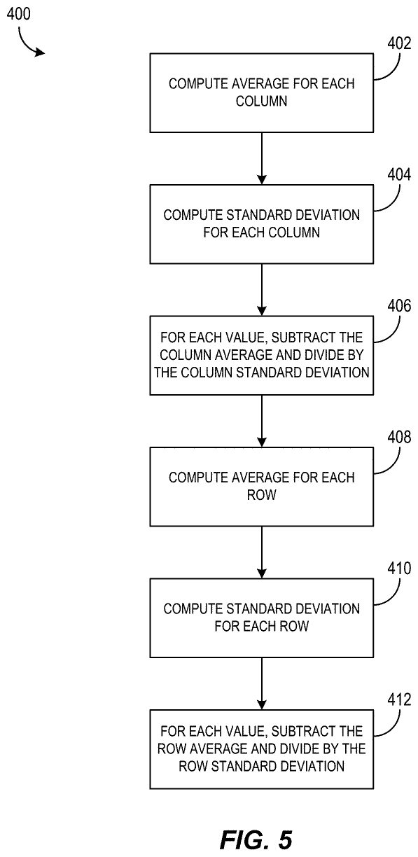

(block 220). Of course, where the expected value E[n_{Xm}] for the count nxm is specific to the individual, in addition to the column and the nucleotide type, the determined expected value for the count will be E[N_{jkXm}], while where the expected value E[n_{Xm}] for the count n_{Xm} is not specific to the individual, the determined expected value for the count will be E[N_{kXm}]. Put another way, when retrieving from memory the expected value E[n_{Xm}] for the count n_{Xm}, the processor 108 will retrieve a value from a three-dimensional data structure with dimensions j, k, and m, where the expected value E[n_{Xm}] for the count n_{Xm} is dependent on the specific individual, or will retrieve a value from a two-dimensional data structure with dimensions k and m, where the expected value E[n_{Xm}] for the count nxm is not dependent on the specific individual. Methods for determining the expected values E[n_{Xm}] are described below with reference to FIGS. 4A and 4B.

The value ΔXₘ is added to a value in a location of the data structure corresponding to column k and row Xₘ (block 222). If m is not equal to w (block 224) (i.e., if blocks 216-222 have not yet been completed for each of the w nucleotide types), the processor 108 increments m (block 226) and completes blocks 216-222 for the next nucleotide type. If m=w (block 224) (i.e., if blocks 216-222 have completed for each of the w nucleotide types), the processor 108 determines if all SNPs have been analyzed (block 228) and, if not, increments i and initializes m to 1 (block 230), before analyzing the next SNP (at block 204).

On other hand, if all SNPs have been analyzed (i.e., if i=y), then the processor 108 determines if there are additional individuals for whom fingerprints should be generated (block 232). If so (i.e., if j is not equal to z), then the processor 108 increments j, and initializes i and m to 1, before starting the next digital fingerprint (at block 204). If not, the method 200 for generating SIMFs is complete (block 236).

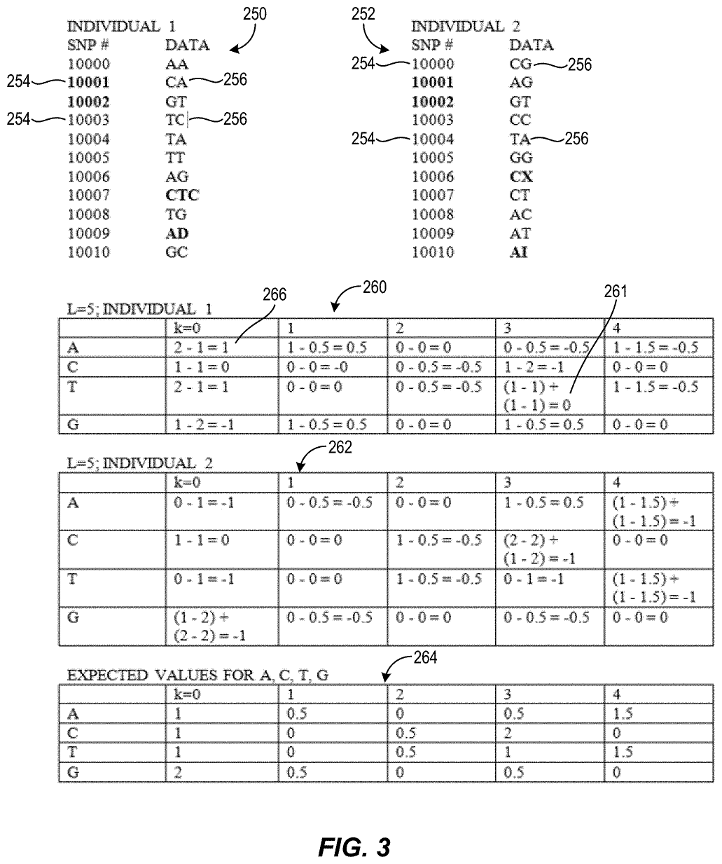

FIG. 3 depicts an extremely simplified example of the data that may be produced by the method 200. In particular, FIG. 3 includes two sets 250, 252 of SNP data corresponding, respectively, to two individuals. Each SNP includes a SNP ID 254 and associated allele data 256. FIG. 3 also includes an associated data structure 260 corresponding to the set 250 of SNP data, and a data structure 262 corresponding to the set 252 of SNP data. Each of the data structures 260, 262 corresponds to an extremely simplified raw SIMF. For the example genotype fingerprints represented by the data structures 260, 262, the following conditions are assumed: vector length, L, is 5; the selected set of SNPs excludes IDs 10001 and 10002; the analysis is limited to biallelic SNPs; and SNPs with nucleotides A, C, T, and G will be analyzed. As a result of these criteria, SNPs 10001 and 10002 will be excluded from both genotype fingerprints, SNPs 10007 and 10009 will be excluded from the genotype fingerprint for individual 1, and SNPs 10006 and 10010 will be excluded from the genotype fingerprint for individual 2.

In the example depicted in FIG. 3, the expected values are assumed to be the same across all individuals, and to vary only according to the column, k, and the nucleotide type. A data structure 264 depicts example expected values E[N_{kXm}]. Each of the data structures 260, 262 is created according to the method 200. For instance, walking through the method 200, the SNP 10000 for individual 1 is in the selected set (block 204), is autosomal (block 206), is biallelic (block 208), and has only nucleotides in the selected set (block 210). The value of k (10000 mod 5) is 0 (block 214). The number of occurrences of nucleotide A is 2 (AA) (block 216). The expected value for A in column 0 is 1, according to the data structure 264 (block 218). ΔX_{A}=2−1=1, as depicted in cell 266 of data structure 260 (block 220). The calculated value ΔX_{A} is added to the value at cell 266 (corresponding to row X_{A} (A), column k (0) (block 222).

Of course, while depicted in data structures 260, 262 showing the calculations (e.g., cell 266 depicts “2−1=1”), the data in the data structures 260, 262 are generally depicted simply as running totals. For example, a cell 268 in the data structure 260 depicts a first tally (“1−1”) and a second tally (“1−1”) both adding up to 0 (0+0=0). The first tally corresponds to SNP 10003 of individual 1 (TC—one instance of T, in column k=3), while the second tally corresponds to SNP 10008 of individual 1 (TG—one instance of T in column k=3). The data structure 264 indicates that the expected value for nucleotide T at column k=3 is 1.

Turning now to FIGS. 4A and 4B, an example method 300 of determining the expected count for nucleotides is described. The method 300 determines an expected count for each nucleotide type m, for each column k, for each individual j, based on the cohort average (i.e., based on the total set of data for the group of genotype fingerprints being computed/created). As such, the method 300 is a prerequisite to the method 200, inasmuch as the expected values for a particular individual require an analysis of the entire cohort.

Within FIGS. 4A and 4B, a particular notation is used that is worth explaining in some detail. As used throughout the specification, N_{jkXm} is the number of occurrences of nucleotide Xₘ in column k for individual j. In the method 300, the depicted flow charts replace an index over which a total is computed by a “+”, which results in the following notations and associated corresponding equations:

Nⱼₖ₊=Σₘ₌₁^{w}N_{jkXm} (Eq. 3)

N_{j+Xm}=Σₖ₌₀^{L-1}H_{jkXm} (Eq. 4)

N_{+kXm}=Σⱼ₌₁^{z}N_{jkXm} (Eq. 5)

N₊ₖ₊=Σⱼ₌₁^{z}Σₘ₌₁^{w}N_{jkXm}=Σₘ₌₁^{w}N_{+kXm} (Eq. 6)

N₊₊₊=Σₖ₌₀^{L-1}N₊ₖ₊ (Eq. 7)

where:
Nⱼₖ₊ is the total number of nucleotides observed in column k for individual j;
N_{+kXm} is the total number of nucleotides observed in column k, for nucleotide type m, across all individuals; and
N₊ₖ₊ is the total number of nucleotides observed in column k across all individuals.
With these notations in mind, then, the frequency of nucleotide type Xₘ in column k, observed across all individuals is:

$$\frac{{N}_{+ k X m}}{{N}_{+ k +}} \qquad ( Eq . 8 )$$

such that the total of frequencies across all nucleotide types Xm is equal to 1:

$${\sum }_{m = 1}^{w} \frac{{N}_{+ k X m}}{{N}_{+ k +}} = 1 \qquad ( Eq . 9 )$$

With the value in Eq. 8 representing the frequency of nucleotide type Xₘ in column k, observed across all individuals, multiplying that value by the total number of nucleotides observed in column kfor individual j, will yield the expected number of nucleotide type Xₘ at column k, for individual j:

$$E [ {N}_{j k X m} ] = {N}_{j k +} \cdot \frac{{N}_{+ k X m}}{{N}_{+ k +}} \qquad ( Eq . 10 )$$

FIGS. 4A and 4B depict in flow chart form the method 300 for calculating the expected counts, based on cohort averaging, of each nucleotide type Xₘ, in each column k, for each individual j. In the method 300, the processor 108 initializes the values of variables (block 302) such that the variables i, j, m, and k, are initialized, respectively, to 1, 1, 1, and 0. The processor 108 then filters the available SNP data (blocks 204-210) to limit the analysis to particular SNPs or types of SNPs, just as in the method 200 described above. For instance, each SNP, SNPᵢ, may be analyzed to determine if the SNP is in a selected set of SNPs for analysis (block 304). That is, a particular implementation of the method 300 may analyze only some of the available SNPs based on the goal of the analysis. If the SNPᵢ is not in the set of SNPs selected for analysis, the processor 108 may increment i (block 312) and begin analysis of the next SNP. If the SNPᵢ is in the set of SNPs selected for analysis, the processor 108 may determine if the SNP is autosomal (block 306). If not, the processor 108 may increment i (block 312) and begin analysis of the next SNP. If so, the processor 108 may determine whether the SNP includes the desired number (e.g., 2) of alleles (block 308). If the SNPᵢ includes more or fewer alleles than desired (e.g., includes 3 or 1 alleles), the processor 108 may increment i (block 312) and begin analysis of the next SNP. Otherwise, the processor 108 may analyze whether the SNP, includes nucleotide types other than those selected (e.g., other than A, C, T, G) (block 310). If the SNP includes additional nucleotide types, the processor 108 may increment i (block 312) and analyze the next SNP. If the SNP includes only the selected nucleotide types, the processor 108 may proceed with the remainder of the method 300.

As with the method 200, above, the processor 108 performing the method 300 need not perform all of the filtering steps (blocks 304-310). In various embodiments, some or all of the filtering steps (blocks 304-310) depicted in FIG. 4A may be omitted. Additionally, while FIG. 4A depicts the filtering steps (blocks 304-310) performed on a SNP by SNP basis during the performance of the method 300, some or all of any implemented filtering steps may performed separately on the total set of SNP data prior to implementation of the method 300 for determining expected nucleotide counts.

In any event, for each SNPᵢ that meets the required criteria established by the filters (blocks 304-310) the SNP is processed to sort its associated data into a data structure for use during the process of determining the expected nucleotide counts. First, the processor 108 determines into which column the data will be sorted by calculating a reduced rsID, k, for the SNPᵢ according to Eq. 1, above (block 314).

The processor 108 then determines the number of occurrences, nxm, of nucleotide type Xₘ in SNPi (block 316), and adds that number to a running tally in the data structure at row Xₘ, column k (block 318). The processor 108 next evaluates whether all of the nucleotide types Xₘ have been tallied (i.e., whether m=w) (block 320) and, if not, increments m (block 322) and returns to block 316. If all of the nucleotide types Xₘ have been tallied (i.e., m=w), then the processor 108 evaluates whether all of the SNPs for the individual have been counted (i.e., whether i=y) (block 324). If not all of the SNPs have been counted, the processor 108 increments i and initializes m to 1 (block 326), and control turns to the filtering steps (blocks 304-310); otherwise, if all of the SNPs for the individual have been counted (i.e., i=y) (block 324), the processor 108 initializes k, m, and N_{+kXm} to 0, 1, and 0, respectively (block 328).

The processor 108 then calculates the total number of nucleotides observed in column k for individual j (N_{j,k+}) for the individual and column, according to Eq. 3 (block 330). Then, the processor 108 adds the number of occurrences of nucleotide Xₘ in column kfor individual j (N_{jkXm}) to the total number of nucleotides observed in column k, for nucleotide type m, across all individuals (N₊ₖₓₘ) (block 332) for each of nucleotide types Xₘ. That is, the processor 108 executes block 332, then determines whether there are additional nucleotide types to analyze (i.e., whether m=w) (block 334) and, if there are additional nucleotide types to analyze (i.e., m is less than w), increments m (block 336) and returns to block 332. If there are no additional nucleotide types to analyze (i.e., m=w), the processor 108 determines whether all columns k have been analyzed (i.e., whether k=L−1) (block 338). If there are additional columns k to analyze, the processor 108 increments k and initializes m to 1 (block 340) and returns control to block 330. If there are no additional columns k to 0 analyze (i.e., k=L−1), then the processor 108 determines whether SNP data for all individuals have been analyzed (i.e., whether j=z) (block 342). If not, the processor 108 increments j and initializes both i and m to 1 (block 344) and returns control to the filtering steps (blocks 304-310).

Referring now to FIG. 4B, if the processor 108 determines that SNP data for all individuals have been analyzed (i.e., j=z) (block 342), the processor 108 initializes k to 0 (block 346). The processor 108 then calculates the total number of nucleotides observed in column k across all individuals (N₊ₖ₊), according to Eq. 6 (block 348), initializes j=1 (block 350) and initializes m=1 (block 352). The processor 108 then calculates the expected number of nucleotide type Xₘ at column k, for individual j, E[Nⱼₖₓₘ], for each nucleotide type Xₘ, individual j, and column k, according to Eq. 10. Specifically, the processor 108 calculates E[N_{jkXm}] for a particular nucleotide type Xₘ, column k, and individual j (block 354), and then evaluates whether there are additional nucleotide types for which E[N_{jkXm}] needs to be evaluated for a specific column k and individual j (i.e., whether m=w) (block 356). If so, the processor 108 increments m (block 358) and returns to block 354. If not, the processor determines whether there are additional individuals for which to calculate values of E[N_{jkXm}] (i.e., whether j=z) (block 360). If so, the processor increments j(block 362) and returns to block 352. If not, the processor determines whether there are additional columns kfor which to calculate E[N_{jkXm}] (i.e., whether k=L−1) (block 364). If so, the processor 108 increments k (block 366) and returns to block 348. If not, the method 300 is complete, having resulted in a three-dimensional data structure of expected values E[Nⱼₖₓₘ] for each column k, nucleotide type Xₘ, and individual j.

It will be understood that, while depicted in a particular order in the flow charts of FIGS. 4A and 4B, the blocks 302-366 need not necessarily be in the precise order in which they are arranged. For instance, without undue experimentation, one could determine other orders of operations that would result in the same set of information.

In embodiments, the method 300 may be omitted entirely where the allele frequencies for each SNP ID are known or calculable over a population significantly larger than the cohort of genotypes being examined. However, calculating allele frequencies in this way can incur significant computational costs.

The methods above result in a raw SNP ID modulo fingerprint. The raw SNP ID modulo fingerprints that result from the methods of FIGS. 3, 4A, and 4B have significant internal structure, both in the scale of the columns and the scale of the rows. The dimension (rows or columns, typically rows) that represents the nucleotide types, can each be affected by the types of nucleotide substitutions (i.e., transitions or transversions) present in a genotype. Because transitions are more common than transversions, allele pairs that arose via transitions are more common than those that arose via transversions. All of the internal structural information is inherent to the method and does not add additional information about the genotypes represented by the SNP ID modulo fingerprints. Accordingly, it may be helpful to remove this non-informative structure by normalizing the SNP ID modulo fingerprint to remove the internal structure.

FIG. 5 depicts an example method 400 for normalizing a raw SNP ID modulo fingerprint generated according to methods FIGS. 3, 4A, and 4B. The normalization method 400 involves computing the average and standard deviation for each column in the matrix (blocks 402 and 404, respectively). Thereafter, a Z-score is computed by subtracting the average value for each column from each value in the column, and dividing the standard deviation for each column into each value in the column (block 406). It should be understood that the Z-score (also known as a standard score) represents the signed number of standard deviations the value is above the mean. The method 400 also involves computing the average and standard deviation for each row in the matrix (blocks 408 and 410, respectively). Thereafter, the average value for each row is subtracted from each value in the row, and standard deviation for each row is divided into each value in the row (block 412).

It will be appreciated that additional utility may be obtained by adjusting fingerprints for population (e.g., ethnic or otherwise) to remove biases toward European (or other) populations that may be present in the reference genome(s) (e.g., the freeze or freezes from which initial representations are generated). For instance, the SNP ID modulo fingerprints may be better sensitized to recognizing the relatedness of individuals if the SNP ID modulo fingerprints are normalized to the population to which the individual(s) belong.

In principle, a “population” for purposes of adjusting or normalizing can be selected based on any selected trait or traits. In some variations, the population is selected based on a phenotypic trait, e.g., a disease condition or physical attribute. In some variations, the population is selected based on geographic origin, ethnicity, race, sex, or other criteria. If established scientific criteria do not exist for defining the population, then individuals can be classified by whether they self-identify as a member of the population, e.g., using a questionnaire.

A method 420 for adjusting SNP ID modulo fingerprints for population is depicted in FIG. 6. Generally speaking, the method 420 involves generating a population fingerprint for the population in question. The population fingerprint is actually two matrices—a first matrix comprising averages, and a second matrix comprising standard deviations. Thus, for each value in the SNP ID modulo fingerprint, the average is computed over a set of many SNP ID modulo fingerprints from the population in question (block 422) to generate a matrix of averages, and the standard deviation is computed over the same set of many SNP ID modulo fingerprints (block 424) to generate a matrix of standard deviations. To perform population adjustment on a particular SNP ID modulo fingerprint, then, each value in the SIMF may be adjusted by subtracting from the value the corresponding average (taken from the matrix of population averages) and dividing it by the corresponding standard deviation (taken from the matrix of population standard deviations) (block 426). In an alternative embodiment (not shown), the computation at block 424 is not implemented such that no matrix of standard deviations is generated. In the alternative embodiment, method 420 is simplified, requiring only generation of the matrix of averages (block 422) and performing the population adjustment on a particular SNP ID modulo fingerprint by adjusting each value in the SIMF by subtracting from the value the corresponding average (taken from the matrix of population averages). Other alternative embodiments are further possible, including subtracting a matrix of medians (instead of a matrix of averages, as described above), subtracting a matrix of medians and dividing by a matrix of median absolute deviations (instead of a matrix of standard deviations, as discussed above). Moreover, in some embodiments, population adjustment is performed on a SNP ID modulo fingerprint that has been previously normalized according to the method 400.

It is worth noting that SNP ID modulo fingerprints are only directly comparable when computed using the same vector length, L; different values of L cause SNP IDs to be grouped into columns differently. However, different versions of a genotype array design contain substantial overlaps in the set of SNPs included in the array, and SNP IDs are grouped in the same manner for a given value of L regardless of array design. Thus, genotypes from the same population on slightly different variants of the same array design may be mixed in computing the population fingerprint.

The SNP ID modulo fingerprints may be readily compared with minimal computation requirements and, of course reduced memory requirements relative to complete genotypes. The SNP ID modulo fingerprints generated by the methods 200 and 300 will generate be represented and/or stored as matrices of values, each value representing a variation from an expected number of occurrences of an allele occurs within a group of SNP IDs (i.e., the SNP IDs modulo the vector length). Accordingly, each matrix has dimensions dictated by the number of nucleotide types and the vector length, and each value may be represented by an 8-bit, 16-bit, or 32-bit integer or floating point value. (Of course, there is no requirement that the values be represented by any specific number of bits, so long as the number of bits used is sufficient to represent the required values.) In embodiments, genotype fingerprints are compared that were generated using the same vector length, the same choice of set of nucleotides (including the order of the nucleotides), and/or the same configuration of rows vs. columns (i.e., columns represent “bins” in both fingerprints or rows represent “bins” in both fingerprints.

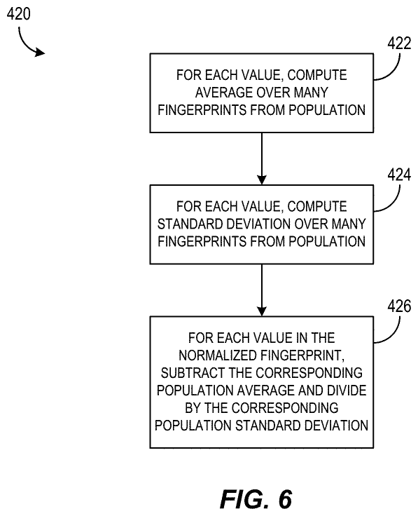

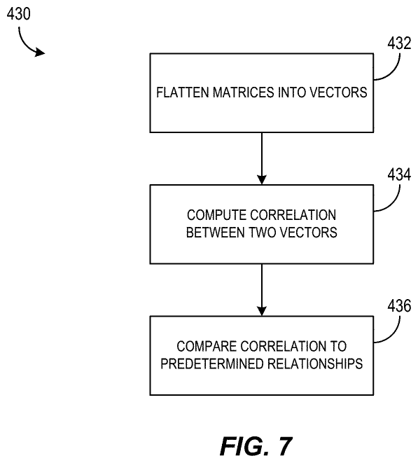

FIG. 7 depicts an example method 430 of comparing SNP ID modulo fingerprints. Two SNP ID modulo fingerprints may be compared to one another by first flattening the matrix representing each SIMF to a vector (block 432). This may be accomplished, for example, simply by concatenating the rows of each of the matrix, such that each matrix is transformed into a corresponding vector. Computing a correlation—for example a Spearman correlation—between the two vectors (block 434) will allow the vectors and the corresponding genotypes to be compared to determine one or more of a variety of characteristics, as described below, by comparing the correlation between the two vectors to various predetermined relationships (block 436). (Other types of correlations can also be used. Two such other correlations are the Pearson correlation and the Kendall correlation.)

Of course, as alluded to above, the sensitivity of the methods and systems described herein, and the utility of the embodiments implementing different sensitivities, may be varied in a variety of ways. As described above, it is possible to adjust the sensitivity of the method and/or system by adjusting the vector length parameter to adjust the sensitivity of the method. For instance, SNP ID modulo fingerprints generated using a vector length of 5000 may perform quite well for determining close family relationships, but may or may not perform as well for population analyses. Population analyses may experience better performance from distance modulo fingerprints generated with a vector length of around 10000, for example.

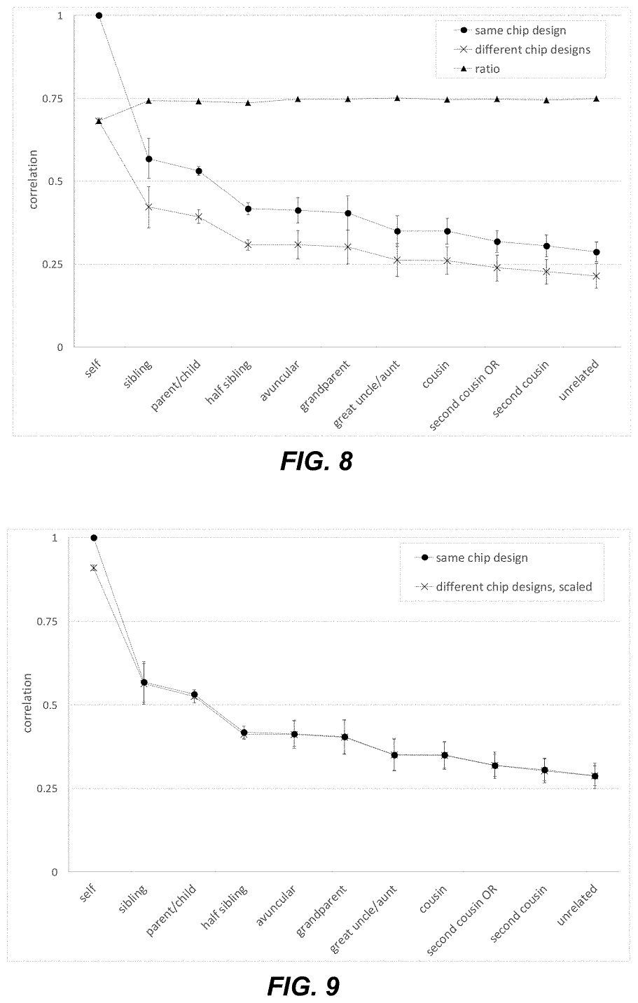

FIGS. 8 and 9 demonstrate that the contemplated embodiments of the genotype fingerprints provide consistent comparability between SNP ID modulo fingerprints regardless of whether the genotype data for two SIMFs are generated by the same SNP chip design or by different SNP chip designs. FIG. 8 depicts, for a studied set of genotype fingerprints for which relationships were known a priori, the correlations for each type of relationship, both when the compared genotype fingerprints were created from genotypes generated by the same SNP chip design and when the compared genotype fingerprints were created from genotypes generated by different SNP chip designs. As the data show, the difference in correlation values for a given relationship maintains a generally consistent ratio between same-chip design comparisons and different-chip design comparisons. As illustrated in FIG. 8, in the example studied to determine the depicted data, that ratio is approximately 0.75. Of course, in other samples, that ratio may be different, but can be determined and applied in much the same way. By using that ratio as a correction factor, it is easy to see that, even across different SNP chip designs, the present methods/systems facilitate comparison of genotypes and identification of familial relationships among them, as demonstrated in FIG. 9.

Of course any and/or all of the methods described above, including the methods 200, 300, 400, 420, and/or 430, may be executed by systems comprising a computer (e.g., the computer 100) that may or may not be communicatively coupled to a network (e.g., the network 118) and/or to other servers (e.g., the server 120) and/or databases (e.g., the database 122). The methods 200, 300, 400, 420, and/or 430 may be embodied as one or more applications, routines and/or modules stored on tangible, non-transitory, computer-readable media (e.g., the memory 110) such that a processor (e.g., the processor 108) may retrieve the instructions for execution. The instructions may be embodied and/or stored as one or more modules, routines.

In various embodiments, databases and related computer-implemented tools, such as online websites and webpages, may be created and implemented to store and provide access to genotype fingerprints. In some embodiments, the database may be private, for example, accessible to only those with specific security permissions. In other embodiments, the database may be made public, for example, accessible to anyone. In some embodiments, the database may be implemented as one or more online databases accessible via a computer network, for example, database 122 associated with server 120 and accessible via network 118, as shown in FIG. 1.

Need for such database-centric solutions arises as the number of known genotypes expands, such that genotype management, identification, and analysis has become drastically more complex. In some embodiments, a database of genotype fingerprints may be used to determine which individuals have been recruited in multiple studies or to find cryptic relatedness in study populations that will cause statistical issues. In other aspects a fingerprint based database may be used to provide answers to common genotype analysis questions, including, for example, determining whether a certain genotype has been seen before; whether similar genotype have been seen before; whether genotype of relatives have been seen; or what genotype or genotypes are most similar, at least with respect to those genotypes stored in the database.

The database may be part of a fingerprint management system. The use of the management system, for example, could allow researchers to manage data from large numbers of genotypes through fingerprints. For example, a public database of genotype fingerprints can support several applications (e.g., study design “matchmaking”), while maintaining privacy. In another aspect, the database may store and provide a method for computing personalized allele frequencies without requiring prior knowledge of populations.

In other aspects, the fingerprint management system may provide open source tools for implementing local, private fingerprint databases. In such an aspect, researchers installing a local copy of the management system are able to directly use genotype fingerprinting in their research.

In other aspects, a public database of genotype fingerprints may be used, the public databases using an authorization and authentication model to mitigate privacy concerns, but at the same time making all fingerprints available to facilitate creating and study populations easier, population identification faster, and to allow more collaboration in the research community via “data matchmaking.”

In other aspects, the accumulation of known genotypes (with associated fingerprints) in databases allows analyses not previously possible. In particular, the combination of the public genotype fingerprint database with large databases of known genotypes, enables the computation of precise, personalized allele frequencies and genotype frequencies.

As described above, in certain embodiments, computer-implemented tools are disclosed for creating private fingerprint databases. For example, the fingerprint management system, as described herein, can allow for organization of fingerprints for creating fingerprints of various sizes and normalization levels, quickly querying those fingerprints, and running analyses on subsets of fingerprints.

In one embodiment, the fingerprint management system may be an executable file or set of files, program or programs, or code able to be installed and used on a variety of computing operating systems (e.g., Linux systems, Microsoft systems, Apple systems, etc.).

In other aspects, the files or code may be open source code made available from a public repository under a particular code library.

In other aspects, the fingerprint system may support the indexing of multiple sizes of fingerprints and different normalization versions to support the development of algorithms and data exploration, to offer multi genomic analysis results, and provide visualizations of collections of fingerprint data.

For example, a specific online embodiment may include creating an Amazon Web Services (AWS) Lambda function (aws.amazon.com/lambda) as a NodeJS (e.g., a specific JavaScript runtime environment) deployment package that can be used to easily translate genomic source data into fingerprints that are stored on the researcher's Amazon S3 AWS account. In such an implementation, the fingerprint database system may use a modular architecture based on microservices, as described in [CITE Bahsoon 2016], which is incorporated by reference herein.

In the specific embodiment, the database may be built using, for example, the “MEAN” software stack (MongoDB, Express, Angular2, NodeJS) with frontend visualizations using D3 (d3js.org) and a REST (Representational state transfer) API backend as a scalable high availability web service.

The MongoDB (i.e., a NoSQL based database implementation) may be used to store and support expansion to hundreds of thousands of genotype fingerprints. To support scaling to millions of genotypes, alternative solutions may be used, including in-memory data stores like (e.g., Redis (redis.io)) and distributed graph databases such as Titan (titan.thinkaurelius.com).

In various embodiments, as described herein, a public genotype fingerprint database may be created. In some aspects, the public fingerprint database may facilitate creation of study populations, genomic analysis, and matchmaking between researchers. However, such public availability of fingerprint information may raise significant privacy concerns, e.g., metadata about particular fingerprints could be used to create likely matches to clinical data already possessed by a researcher. Accordingly, as described further herein, in one embodiment, a public genotype fingerprint database may be characterized and add data in three stages: Public Data, Private Data, and Federation, with each data level designating a particular privacy or security level.

In Stage 1, the genotype fingerprint database includes only fingerprints computed from Public Data, defined as sets of genomes that any qualified individual can obtain freely for research purposes.

In Stage 2, the database also includes fingerprints computed from Private Data as submitted by researchers. The privacy requirements for the private data fingerprints may be defined, such that addition of the fingerprints to the database required the fingerprints to meet a specific level of privacy or authorization.

In some aspects, data access to the database is granular, with each attribute of a resource and its metadata having individual permissions or residing as part of a group policy. Community researchers who submit fingerprints to the database are able to select an authorization level for their data and provide their contact information and select from several methods for requesting data access. The private fingerprint database may use data authentication and authorization to protect the system and keep the information private.

In a specific embodiment, use of a public identity provider, such as provided by Google, Amazon, or Auth0, allows users to create accounts to access the private data available on the fingerprint server. Such a system may be modeled around the Amazon Identity Access and Management (IAM) system, with users able to be assigned to groups and assume roles with specific permissions.

In certain aspects, different data authorization categories may be offered, e.g.: Public, Institution, Registered, and Private. Public authorization requires login with a public identity provider only. Institution authorization requires login with a specific institution's identity provider. Registered authorization requires login with an identity provider and a registered access attestation. Private authorization means that the user will receive information that there is a match in the database and the fingerprint identifier, but no access to the fingerprint and contact information for a researcher depending on the method selected by that researcher.

In some aspects, a user of the database system may select methods of contact. For example, a user may select the following methods to be contacted by another user: Website, Email, Phone, and Anonymous Message. In other aspects, the contact may be used to approve access requests. For example, once a user is contacted, the user can approve a request by another user by adding specific permissions for the other user or by adding the other user to a group or broader security policy.

In other aspects, and at the highest level of data restriction, a particular user may receive information informing that a match (within a specified threshold) has been found. The user may then send an anonymous message to the owner or researcher associated with the data, requesting more information. For this purpose, such private data may be stored on an encrypted microservice that may use policies or certificates to determine authorization for retrieval of matches and creation of contact requests.

In Stage 3, the database may have a Federation model that supports distributed queries into fingerprint databases stored at other institutions. The Federation model may allow sharing fingerprint databases and related data. For example, the Federation model allows fingerprint databases to communicate with each other so that a query to any connected fingerprint database can return results from all connected fingerprint databases based on the level of sharing selected.

In some embodiments, sharing modes are implemented. For example, Basic sharing mode allows requests that can return a yes/no result, Similarity sharing mode can return the fingerprint identifier and similarity match, and Full sharing mode can return the fingerprint identifier, similarity match, and fingerprint of specified size, subject to authorization and authentication restrictions, as described herein.

In other aspects, databases may store fingerprints to allow researchers or others to compute correlations between individuals with the goal of computing personalized allele frequencies, as described herein.

The methods and systems described herein have a number of advantages over prior methods and systems for performing analysis of genotype sequences. As already discussed, the methods and systems are agnostic to, and do not require knowledge of, the technology, reference, and encoding used to generate the genotype information, which means that the same methods can be used on databases containing sets of data generated using disparate SNP chip technologies. Storage requirements for the data related to individual genotypes is significantly reduced and, accordingly, large data sets require significantly smaller quantities of memory. Further, computation performed on the genotype fingerprints is also faster (i.e., than other computations performed on the same processor) and requires significantly less memory.

Privacy is another benefit of the SIMFs described herein. Because the SIMFs retain only information about the frequency of various alleles in a particular set of SNP IDs (i.e., the set reduced into a particular column), it is essentially impossible to reconstruct from a SIMF the original genotype, with shorter vector lengths being more effective for obscuring genotype data and preventing reverse-engineering. As a result, it is difficult or impossible to identify or predict phenotypes associated with a particular SIMF. Nor is it possible to identify a specific individual from a SIMF alone; such identification can only be made in the context of comparing a SIMF to a SIMF that has been previously prepared for the individual.

The SIMFs described throughout this specification have a variety of uses including, by way of example and without limitation:

Simplifying the size and complexity of data required to uniquely characterize an individual's genotype and differentiate it from genotypes of other individuals of the same species;

Simplifying the size and complexity of data required to maintain library or database of individual genotypes in a format that permits searching or querying or comparing, which has applications in all scientific and other fields (forensics; law enforcement) in which the maintenance and querying of a genotype database for matches may be desirable;

Combining genotype datasets more easily and in a manner that more readily facilitates identification and elimination of duplicate entries;

Establishing whether two genotype representations are derived from the same individual—regardless of genotyping technology;

Establishing whether two genotype representations are derived from closely related individuals;

Testing whether a new genotype has already been observed (e.g., by comparing to a growing database of SIMFs;

Querying a genotype database to determine whether a query genotype is present and/or whether a parent, sibling, grandparent, cousin, or other close relative's genotype is present;

Testing for shared genotypes in two or more studies;

Identifying population(s) of origin by comparing individual fingerprints to population fingerprints;

Selecting matched genotypes by populations (e.g., finding most relevant control data, nearest neighbor search, etc.);

Computing kinship matrices from a collection of genotypes, useful for performing genotype-wide association studies—removing a significant computational bottleneck;

Accelerating population structure studies by computing on a reduced representation of the genotypes; and

Detecting gross chromosomal abnormalities by, for example, computing chromosome-specific SIMFs.

Throughout this specification, plural instances may implement components, operations, or structures described as a single instance. Although individual operations of one or more methods are illustrated and described as separate operations, one or more of the individual operations may be performed concurrently and, unless specifically described or otherwise logically required (e.g., a structure must be created before it can be used), nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

For example, the network 118 may include but is not limited to any combination of a LAN, a MAN, a WAN, a mobile, a wired or wireless network, a private network, or a virtual private network. Moreover, while only one computer 100 is illustrated in FIG. 1 to simplify and clarify the description, it is understood that any number of computers 100 are supported and can be in communication with the server or servers 120 and/or the database or databases 122.

Additionally, certain embodiments are described herein as including logic or a number of components, modules, routines, applications, or mechanisms. Applications or routines may constitute either software modules (e.g., code embodied on a machine-readable medium or in a transmission signal) or hardware modules. A hardware module is tangible unit capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently or semi-permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions.

Similarly, the methods or routines described herein may be at least partially processor-implemented. For example, at least some of the operations of a method may be performed by one or processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

The one or more processors may also operate to support performance of the relevant operations in a “cloud computing” environment or as “software as a service” (SaaS). For example, at least some of the operations may be performed by a group of computers (as examples of machines including processors), these operations being accessible via a network (e.g., the Internet) and via one or more appropriate interfaces (e.g., application program interfaces (APIs)).

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Unless specifically stated otherwise, discussions herein using words such as “processing,” “computing,” “calculating,” “determining,” “presenting,” “ displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description should be read to include one or at least one and the singular also includes the plural unless it is obvious that it is meant otherwise.

Still further, the figures depict preferred embodiments of a system and methods for generating and comparing distance modulo fingerprints for purposes of illustration only. One skilled in the art will readily recognize from the preceding discussion that alternative embodiments of the structures and methods illustrated herein may be employed without departing from the principles described herein.

Upon reading this disclosure, those of skill in the art will appreciate still additional alternative structural and functional designs for a system and a process for generating and comparing SNP ID modulo fingerprints through the disclosed principles herein. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

The following list of aspects reflects a variety of the embodiments explicitly contemplated by the present application. Those of ordinary skill in the art will readily appreciate that the aspects below are neither limiting of the embodiments disclosed herein, nor exhaustive of all of the embodiments conceivable from the disclosure above, but are instead meant to be exemplary in nature.

1. A computer-implemented method of generating a genotype fingerprint representing a genotype, the method comprising: receiving genotype data for an individual, the genotype data including a plurality of single-nucleotide polymorphisms (SNPs), each SNP having an associated identification number (ID) indicating a corresponding location in a genome of the individual, and each SNP having a defined number of alleles each selected from a defined set of nucleotide types; selecting a subset of SNPs from the plurality of SNPs; for each SNP in the subset of SNPs: determining a value (k) associated with the SNP, the value indicating a column of a data structure, and determined by computing the modulus of the identification number divided by a vector length (L), such that k=ID mod L; for each nucleotide type of the defined set of nucleotide types: determining a number of occurrences of the nucleotide type in the SNP, the nucleotide type corresponding to a row of the data structure; determining an expected count in the SNP for the nucleotide type; calculating a difference between the number of occurrences of the nucleotide type in the SNP and the expected count in the SNP for the nucleotide type; adding the difference to a tally at a position in the data structure corresponding to both the column and the row.

2. A computer-implemented method according to aspect 1, wherein the identification number is an rsID number.

3. A computer-implemented method according to either aspect 1 or aspect 2, wherein the determined number of alleles is two.

4. A computer-implemented method according to any one of aspects 1 to 3, wherein the defined set of nucleotide types consists of A, C, T, and G.

5. A computer-implemented method according to any one of aspects 1 to 4, wherein the plurality of SNPs include only autosomal SNPs.

6. A computer-implemented method according to any one of aspects 1 to 5, wherein the data structure comprises one row for each of the defined set of nucleotides, and a number of columns equal to the vector length (L).

7. A computer-implemented method according to any one of aspects 1 to 5, wherein the rows and columns are reversed such that the value (k) associated with the SNP indicates a row of the data structure, and the nucleotide type corresponds to a column of the data structure.

8. A computer-implemented method according to any one of aspects 1 to 7, wherein the vector length (L) is less than the number of SNPs in the selected subset of SNPs.

9. A computer-implemented method according to any one of aspects 1 to 7, wherein the vector length (L) is selected such that the number of SNPs in the selected subset of SNPs is at least 20 times L.

10. A computer-implemented method according to any one of aspects 1 to 9, wherein determining an expected count in the SNP for the nucleotide type comprises computing a cohort average.

11. A computer-implemented method according to aspect 10, wherein computing a cohort average comprises determining, by analyzing a number of genotypes, an average number of occurrences of each nucleotide type for each column for the number of genotypes.

12. A computer-implemented method according to aspect 11, wherein cohort average is weighted, for each genotype, by the number of alleles contributed by that genotype.

13. A computer-implemented method according to any one of aspects 1 to 10, wherein determining an expected count in the SNP for the nucleotide type comprises: calculating, for each of a plurality of individuals, an expected count for each of the nucleotide types in each column, such that: E[Nⱼₖₘ]=Nⱼₖ₊*(N₊ₖₘ/N₊ₖ₊) where: E[Nⱼₖₘ] is the expected number allele m, at column k, for individual j, Nⱼₖ₊ is the total number of nucleotides observed, for individual j, at column k, N₊ₖₘ is the total number of nucleotides observed, across all individuals, at column k, for a particular nucleotide type, N₊ₖ₊ is the total number of nucleotides observed, across all individuals, for all nucleotide types, at column k, and (N₊ₖₘ/N₊ₖ₊) is the frequency of allele m, at column k, across all individuals, such that the sum of (N₊ₖₘ/N₊ₖ₊) across all alleles is equal to 1.

14. A computer-implemented method according to any one of aspects 10 to 13, wherein the genotype data for each of the plurality of individuals is determined by a SNP chip analysis, and wherein the computer-implemented method generates a genotype fingerprint for each individual's genotype.

15. A computer-implemented method according to aspects 14, wherein the SNP chip analysis for a first subset of the plurality of individuals is determined according to a different SNP chip type or version than the SNP chip analysis for a second subset of the plurality of individuals, and wherein the method results in genotype fingerprints for which relatedness of the two individuals can be determined even when the genotype data for a first of the two individuals was determined by a first SNP chip type or version and the genotype data for a second of the two individuals was determined by a second SNP chip type or version.

16. A computer-implemented method according to any one of aspects 1 to 15, further comprising: representing the genotype fingerprint as a matrix; and normalizing the matrix relative to a reference matrix derived from a set of genotypes.

17. A computer-implemented method according to aspect 16, wherein normalizing the matrix relative to the reference matrix comprises: representing each genotype fingerprint of the set of genotype fingerprints as a corresponding matrix; computing, for each position of the matrix, an average and a standard deviation for each matrix in the set of matrices from which the reference matrix is derived; and transforming the matrix by computing a Z-score for each value in the matrix, wherein the Z-score is the value, minus the average, divided by the standard deviation.

18. A computer-implemented method according to either aspect 16 or aspect 17, wherein the set of genotypes is a set of genotypes from an identified population.

19. A computer-implemented method according to any one of aspects 1 to 18, further comprising: representing the genotype fingerprint as a matrix; and normalizing the matrix internally.

20. A computer-implemented method according to aspect 19, wherein the data structure comprises one row for each of the defined set of nucleotides, and a number of columns equal to the vector length (L), and wherein normalizing the matrix internally comprises: computing a column average for each column in the matrix; computing a column standard deviation for each column in the matrix; for each value, subtracting the column average and dividing by the column standard deviation; computing a row average for each row in the matrix; computing a row standard deviation for each row in the matrix; and for each value, subtracting the row average and dividing by the row standard deviation.

21. A computer-implemented method according to aspect 19, wherein the data structure comprises one column for each of the defined set of nucleotides, and a number of rows equal to the vector length (L), and wherein normalizing the matrix internally comprises: computing a row average for each row in the matrix; computing a row standard deviation for each row in the matrix; for each value, subtracting the row average and dividing by the row standard deviation; computing a column average for each column in the matrix; computing a column standard deviation for each column in the matrix; and for each value, subtracting the column average and dividing by the column standard deviation.

22. A computer-implemented method for comparing genotype information, the method comprising: computing, according to the method of any one of aspects 1 to 21, a first genotype fingerprint and a second genotype fingerprint; and determining a correlation between the first genotype fingerprint and the second genotype fingerprint.

23. A computer-implemented method according to aspect 22, wherein determining a correlation between the first genotype fingerprint and the second genotype fingerprint comprises determining a Spearman correlation coefficient.

24. A computer-implemented method according to aspect 22, wherein determining a correlation between the first genotype fingerprint and the second genotype fingerprint comprises determining a Pearson correlation coefficient.

25. A computer-implemented method according to aspect 23, further comprising comparing the Spearman correlation coefficient, p, to one or more thresholds to determine a relationship between respective samples from which the genotype data of the first and second genotype fingerprints were obtained.

1. A computer-implemented method of generating a genotype fingerprint representing a genotype, the method comprising:
receiving genotype data for an individual, the genotype data including a plurality of single-nucleotide polymorphisms (SNPs), each SNP having an associated identification number (ID) indicating a corresponding location in a genome of the individual, and each SNP having a defined number of alleles each selected from a defined set of nucleotide types;
selecting a subset of SNPs from the plurality of SNPs;
for each SNP in the subset of SNPs:
determining a value (k) associated with the SNP, the value indicating a column of a data structure, and determined by computing the modulus of the identification number divided by a vector length (L), such that k=ID mod L;
for each nucleotide type of the defined set of nucleotide types:
determining a number of occurrences of the nucleotide type in the SNP, the nucleotide type corresponding to a row of the data structure;
determining an expected count in the SNP for the nucleotide type;
calculating a difference between the number of occurrences of the nucleotide type in the SNP and the expected count in the SNP for the nucleotide type;
adding the difference to a tally at a position in the data structure corresponding to both the column and the row.

2. A computer-implemented method according to claim 1, wherein the identification number is an rsID number.

3. A computer-implemented method according to claim 1, wherein the determined number of alleles is two.

4. A computer-implemented method according to claim 1, wherein the defined set of nucleotide types consists of A, C, T, and G.

5. A computer-implemented method according to claim 1, wherein the plurality of SNPs include only autosomal SNPs.

6. A computer-implemented method according to claim 1, wherein the data structure comprises one row for each of the defined set of nucleotides, and a number of columns equal to the vector length (L).

7. A computer-implemented method according to claim 1, wherein the rows and columns are reversed such that the value (k) associated with the SNP indicates a row of the data structure, and the nucleotide type corresponds to a column of the data structure.

8. A computer-implemented method according to claim 1, wherein the vector length (L) is less than the number of SNPs in the selected subset of SNPs.

9. A computer-implemented method according to claim 1, wherein the vector length (L) is selected such that the number of SNPs in the selected subset of SNPs is at least 20 times L.

10. A computer-implemented method according to claim 1, wherein determining an expected count in the SNP for the nucleotide type comprises computing a cohort average.

11. A computer-implemented method according to claim 10, wherein computing a cohort average comprises determining, by analyzing a number of genotypes, an average number of occurrences of each nucleotide type for each column for the number of genotypes.

12. A computer-implemented method according to claim 11, wherein cohort average is weighted, for each genotype, by the number of alleles contributed by that genotype.

13. A computer-implemented method according to claim 1, wherein determining an expected count in the SNP for the nucleotide type comprises:
calculating, for each of a plurality of individuals, an expected count for each of the nucleotide types in each column, such that:
E[Nⱼₖₘ]=Nⱼₖ₊*(N₊ₖₘ/N₊ₖ₊)
where:
E[Nⱼₖₘ] is the expected number allele m, at column k, for individual j,
Nⱼₖ₊ is the total number of nucleotides observed, for individual j, at column k,
N₊ₖₘ is the total number of nucleotides observed, across all individuals, at column k, for a particular nucleotide type,
N₊ₖ₊ is the total number of nucleotides observed, across all individuals, for all nucleotide types, at column k, and
(N₊ₖₘ/N₊ₖ₊) is the frequency of allele m, at column k, across all individuals, such that the sum of (N₊ₖₘ/N₊ₖ₊) across all alleles is equal to 1.

14. A computer-implemented method according to claim 10, wherein the genotype data for each of the plurality of individuals is determined by a SNP chip analysis, and wherein the computer-implemented method generates a genotype fingerprint for each individual's genotype.

15. A computer-implemented method according to claim 14, wherein the SNP chip analysis for a first subset of the plurality of individuals is determined according to a different SNP chip type or version than the SNP chip analysis for a second subset of the plurality of individuals, and wherein the method results in genotype fingerprints for which relatedness of the two individuals can be determined even when the genotype data for a first of the two individuals was determined by a first SNP chip type or version and the genotype data for a second of the two individuals was determined by a second SNP chip type or version.

16. A computer-implemented method according to claim 1, further comprising:
representing the genotype fingerprint as a matrix; and
normalizing the matrix relative to a reference matrix derived from a set of genotypes.

17. A computer-implemented method according to claim 16, wherein normalizing the matrix relative to the reference matrix comprises:
representing each genotype fingerprint of the set of genotype fingerprints as a corresponding matrix;
computing, for each position of the matrix, an average and a standard deviation for each matrix in the set of matrices from which the reference matrix is derived; and
transforming the matrix by computing a Z-score for each value in the matrix, wherein the Z-score is the value, minus the average, divided by the standard deviation.

18. A computer-implemented method according to c claim 16, wherein the set of genotypes is a set of genotypes from an identified population.

19. A computer-implemented method according to claim 1, further comprising:
representing the genotype fingerprint as a matrix; and
normalizing the matrix internally.

20. A computer-implemented method according to claim 19, wherein the data structure comprises one row for each of the defined set of nucleotides, and a number of columns equal to the vector length (L), and wherein normalizing the matrix internally comprises:
computing a column average for each column in the matrix;
computing a column standard deviation for each column in the matrix;
for each value, subtracting the column average and dividing by the column standard deviation;
computing a row average for each row in the matrix;
computing a row standard deviation for each row in the matrix; and
for each value, subtracting the row average and dividing by the row standard deviation.

21. A computer-implemented method according to claim 19, wherein the data structure comprises one column for each of the defined set of nucleotides, and a number of rows equal to the vector length (L), and wherein normalizing the matrix internally comprises:
computing a row average for each row in the matrix;
computing a row standard deviation for each row in the matrix;
for each value, subtracting the row average and dividing by the row standard deviation;
computing a column average for each column in the matrix;
computing a column standard deviation for each column in the matrix; and
for each value, subtracting the column average and dividing by the column standard deviation.

22. A computer-implemented method for comparing genotype information, the method comprising:
computing, according to the method of claim 1, a first genotype fingerprint and a second genotype fingerprint; and
determining a correlation between the first genotype fingerprint and the second genotype fingerprint.

23. A computer-implemented method according to claim 22, wherein determining a correlation between the first genotype fingerprint and the second genotype fingerprint comprises determining a Spearman correlation coefficient.

24. A computer-implemented method according to claim 22, wherein determining a correlation between the first genotype fingerprint and the second genotype fingerprint comprises determining a Pearson correlation coefficient.

25. A computer-implemented method according to claim 23, further comprising comparing the Spearman correlation coefficient, p, to one or more thresholds to determine a relationship between respective samples from which the genotype data of the first and second genotype fingerprints were obtained.
